(19) United States
(12) Patent Application Publication
(10) Pub. No.: US 2026/0199275 A1
(43) Pub. Date: Jul. 16, 2026

(54) LIQUID COMPOSITION AND THE METHOD THEREOF

(71) Applicant: Panion & BF Biotech Inc., Taipei (TW)

(72) Inventors: Jwey-Yuan Chuang, Taipei (TW); Yu-De Su, Taipei (TW); Yu-Ying Hsu, Taipei (TW); Shu-Yu Chou, Taipei (TW)

(73) Assignee: Panion & BF Biotech Inc., Taipei (TW)

(21) Appl. No.: 19/430,426

(22) Filed: Dec. 23, 2025

Related U.S. Application Data
(60) Provisional application No. 63/738,264, filed on Dec. 23, 2024.

(51) Int. Cl. A61K 31/194; A61K 31/07; A61K 31/19; A61K 31/355; A61K 31/593; A61K 33/10; A61K 47/12; A61P 3/12

(52) U.S. Cl. CPC A61K 31/194; A61K 31/07; A61K 31/19; A61K 31/355; A61K 31/593; A61K 33/10; A61K 47/12; A61P 3/12

(57) ABSTRACT

The present disclosure provides a liquid composition including: ferric citrate, a phosphate binder; and water, wherein a weight ratio of the ferric citrate to the phosphate binder is from 6:1 to 0.9:1. Furthermore, the present disclosure further provides a method for preventing or treating a disease in a subject, including administering to the subject an effective amount of the liquid composition. According to the present disclosure, the ferric citrate and the phosphate binder, in a specific weight ratio, stabilize the pH of the liquid composition and enhance the phosphate binding capacity of the liquid composition.

### CROSS-REFERENCE TO RELATED APPLICATION

This application claims priority to U.S. Provisional Application Ser. No. 63/738,264, filed Dec. 23, 2024, which is herein incorporated by reference in its entirety.

### BACKGROUND

### Field of Invention

The present invention relates to a liquid composition. More particularly, the present invention relates to a liquid composition including ferric citrate, a phosphate binder, and water or oil, and a method thereof for preventing or treating of disease in a subject.

### Description of Related Art

Subjects suffering from chronic kidney disease often develop complications such as hyperphosphatemia because of the incapability of their kidneys to metabolize phosphorus. In order to reduce excess phosphate in the body, phosphate binders such as ferric citrate are commonly used to bind phosphate with ferric ions to form phosphate precipitates, and the excess phosphorus are then excreted from the body. Currently, ferric citrate is mainly administered to the subjects in oral solid dosage form, to achieve the aforementioned objectives. To formulate ferric citrate in a form of oral liquid preparation so that the subjects with difficulty swallowing tablets (patients with dysphagia, or animals such as cats and dogs) may successfully intake ferric citrate is also noticed in this field.

However, the phosphate binding efficiency of ferric citrate in the form of oral liquid preparation may be altered by the components in the solvent, or the stability may be affected. Therefore, determining which components to formulate a ferric citrate oral liquid preparation with improved efficacy remains an ongoing matter in the field.

### SUMMARY

Therefore, in order to meet the needs of the art, the present application provides a liquid composition, which may further include a water-soluble flavoring agent. A pH of the liquid composition is stabilized at 2.5 to 5 and has a phosphate binding effect to form a phosphate precipitate of ferric ions and phosphate.

To achieve the abovementioned objective, the present application provides a liquid composition including: (a) ferric citrate; (b) a phosphate binder; and (c) water.

In one embodiment, a weight ratio of the ferric citrate to the phosphate binder is from 6:1 to 0.9:1.

In one embodiment, the phosphate binder includes a calcium salt of a weak acid.

In one embodiment, the calcium salt of the weak acid includes calcium lactate, calcium glycinate, calcium acetate, calcium carbonate, or a combination thereof.

In one embodiment, in the liquid composition, the ferric citrate and the phosphate binder have a synergistic effect.

In one embodiment, the liquid composition, the ferric citrate and the calcium lactate have a synergistic effect.

In one embodiment, the liquid composition further includes a food additive. The food additive includes: a preservative, an antioxidant, a solubilizer, a flavoring agent, a thickener, or a combination thereof.

In one embodiment, in the liquid composition, the flavoring agent includes a water-soluble flavoring agent.

In one embodiment, in the liquid composition, the flavoring agent is free of propylene glycol.

In one embodiment, the liquid composition includes 1% to 20% by weight of the ferric citrate, based on 100% by weight of the liquid composition.

In one embodiment, the liquid composition includes 1% to 15% by weight of the phosphate binder, based on 100% by weight of the liquid composition.

In one embodiment, a pH of the liquid composition is stabilized at 2.5 to 5.

Another objective of the present application is to provide a method for preventing or treating a disease in a subject, including administering to the subject an effective amount of the liquid composition, wherein the disease includes hyperphosphatemia, metabolic acidosis, or anemia.

In one embodiment, the subject includes a human or an animal.

In one embodiment, the animal includes a dog or a cat.

Another objective of the present application is to provide a liquid composition, including ferric citrate, a phosphate binder, and an oil, wherein a weight ratio of the ferric citrate to the phosphate binder is from 6:1 to 0.9:1.

In one embodiment, the phosphate binder includes a calcium salt of a weak acid.

In one embodiment, the calcium salt of the weak acid includes calcium lactate, calcium glycinate, calcium carbonate, calcium acetate, or a combination thereof.

In one embodiment, further including vitamin A, vitamin D, vitamin E, or a combination thereof.

In one embodiment, the oil includes fatty acid.

### BRIEF DESCRIPTION OF THE DRAWINGS

FIG. 1 is a flowchart of a preparation process of a liquid composition according to some embodiments.

FIG. 2 is a graph showing binding effects of the liquid composition with phosphate according to some embodiments.

FIG. 3 is a graph showing binding effects of phosphate binders alone with phosphate according to some embodiments.

FIG. 4A and FIG. 4B are graphs showing phosphate binding effects of calcium lactate with different flavoring agents, respectively, according to some embodiments.

FIG. 4C and FIG. 4D are graphs showing phosphate binding effects of calcium glycinate and calcium carbonate, respectively, according to some embodiments.

FIG. 4E is a graph showing phosphate binding effects of calcium acetate, according to some embodiments.

### DETAILED DESCRIPTION

Unless otherwise defined, the technical and scientific terms used in the present application are equivalent to the meaning as commonly understood by one of ordinary skill in the art. In the event of any conflict, the definitions included herein shall prevail.

When the percentages are mentioned in the present application, the terms “wt %” and “%” used herein may refer to mass fractions, which are defined as: the mass of a substance divided by the total mass of the mixture, expressed as percentage.

As used herein, the terms “produced from” and “comprising” have the same meaning. As used herein, the terms “includes”, “including”, “comprises”, “comprising”, “has”, “having”, “contains”, “containing” or any variants thereof are intended to cover a non-exclusive inclusion. For example, a composition, process, method, product, or apparatus that contains a list of elements does not necessarily contain only those elements, but may also contain other elements not expressly listed but are inherent to the composition, process, method, product, or apparatus. In general, the term “include” is used in the sense of inclusion, which allows the presence of one or more other features or components.

The indefinite articles “a” and “an” as used in the present application preceding elements or ingredients are intended to provide a non-restrictive description of the quantity of instances (i.e., the number presented) of the elements or ingredients. Therefore, the indefinite articles “a” or “an” should be interpreted as including one or at least one, and the singular form of the elements or ingredients may also be interpreted to mean “including a plurality of”, unless the number obviously refers to the singular.

The objective of the present application is to provide a liquid composition, including: (a) ferric citrate; (b) a phosphate binder; and (c) water.

In some embodiments, a weight ratio of ferric citrate to phosphate binder is from 6:1 to 0.9:1, for example, may be from 5:1 to 0.9:1, may be from 4.9:1 to 0.9:1, may be from 3.3:1 to 0.9:1, may be from 3:1 to 0.9:1, may be from 2.9:1 to 0.9:1, may be from 2.7:1 to 0.9:1, may be from, 2.6:1 to 0.9:1, may be from 2.5:1 to 0.9:1.

In some embodiments, the phosphate binder includes a calcium salt of a weak acid. The term “a calcium salt of a weak acid” as used in the present application refers to salts composed of calcium cations and incompletely dissociated acid radical anions, including but not limited to: calcium lactate, calcium glycinate, calcium acetate, calcium carbonate, or combinations thereof.

The term “phosphate binder” as used in the present application refers to any component, other than ferric citrate, that provides phosphate binding capacity. The embodiment further illustrates that different phosphate binders may be used in combination with ferric citrate, and their weight ratios may be adjusted, so that the pH of the liquid composition is stabilized at 2.5 to 5, achieving a phosphate binding effect.

In some embodiments, the ferric citrate and the phosphate binder have a synergistic effect. In some embodiments, the phosphate binder includes calcium lactate, calcium glycinate, calcium carbonate, or a combination thereof, and the synergistic effect is achieved when a weight ratio of the ferric citrate to the phosphate binder is from 5:1 to 1:1 (for example, 5:1, 4.5:1, 4:1, 3.5:1, 3:1, 2.5:1, 2:1, 1.5:1, 1:1, or any value between any two of these values).

The “synergistic effect” in the present application refers to that when two or more substances are mixed, the effect produced (i.e., phosphate binding effect) is equal to or greater than the sum of the effects produced by each individual substance. For example, as described in the embodiments of the present application, when each of the phosphate binders is used alone without the ferric citrate, the phosphate binding effect of the calcium lactate experimental group is the worst. However, when the ferric citrate is used with the calcium lactate as a liquid composition, the phosphate binding effect is the best, indicating that ferric citrate and calcium lactate have a synergistic effect.

In some embodiments, the liquid composition further includes a food additive. In one embodiment, the food additive includes: a preservative, an antioxidant, a solubilizer, a flavoring agent, a thickener, or a combination thereof. In one embodiment, the preservative includes potassium sorbate.

In one embodiment, the flavoring agent includes a water-soluble flavoring agent. When the solvent of the liquid composition is water, the water-soluble flavoring agent may be uniformly mixed with the liquid composition without adding additives (such as surfactants). In some embodiments, the liquid composition does not include surfactants, as described in the embodiments herein, to avoid affecting the phosphate binding capacity of the liquid composition. In one embodiment, the liquid composition does not include propylene glycol to avoid toxicity to certain animals (such as cats or dogs).

In some embodiments, the liquid composition includes 1% to 20% by weight of the ferric citrate, based on the total weight of the liquid composition. For example, the liquid composition includes 16%, 10%, 5%, or 1% by weight of the ferric citrate. For example, 1%, 2%, 3%, 4%, 5%, 6%, 7%, 8%, 9%, 10%, 11%, 12%, 13%, 14%, 15%, 16%, 17%, 18%, 19%, 20%, or any value between any two of these values.

In some embodiments, the liquid composition includes 1% to 15% by weight of the phosphate binder, based on the total weight of the liquid composition. The weight percentage of the phosphate binder may be referred to in the embodiments of the present application, which is the weight percentage required for a pH of the liquid composition to be stabilized at 2.5 to 5. For example, 1%, 2%, 3%, 4%, 5%, 6%, 7%, 8%, 9%, 10%, 11%, 12%, 13%, 14%, 15%, or any value between any two of these values.

In some embodiments, a pH of the liquid composition is stabilized at 2.5 to 5. For example, the pH includes 2.5, 2.6, 2.7, 2.8, 2.9, 3.0, 3.1, 3.2, 3.3, 3.4, 3.5, 4.0, 4.5, 5.0, or any value between any two of these values. In one embodiment, a pH of the liquid composition is stabilized at 2.8 to 3.5. Within this pH range, the growth of pathogenic microorganisms (such as Escherichia coli) may be inhibited, and the palatability of the liquid composition for certain animals (such as cats or dogs) may be improved.

Another objective of the present application is to provide a method of preventing or treating disease in a subject, including administering to the subject an effective amount of the liquid composition as mentioned above.

The term “effective amount” as used in the present application refers to the amount of an active ingredient or agent (such as a liquid composition) that achieves the desired preventive or therapeutic outcome, for example, for preventing or treating disease in a subject (such as hyperphosphatemia, metabolic acidosis, or anemia). The effective amount of the liquid composition may, to a certain extent, reduce, prevent, inhibit, or alleviate diseases caused by abnormal phosphorus metabolism of the subjects.

Another objective of the present application is to provide a use of the abovementioned liquid composition in the manufacture of a medicament for preventing or treating disease in a subject.

In some embodiments, the subject includes a human or an animal.

In some embodiments, the animal includes a dog or a cat.

In some embodiments, the disease includes hyperphosphatemia, metabolic acidosis, or anemia. The disease to which the liquid composition is applicable may include any disease in which a subject is unable to metabolize phosphorus normally in the body.

Another objective of the present application is to provide a liquid composition, including: (a) ferric citrate; (b) a phosphate binder; and (c) an oil.

In some embodiments, the liquid composition further includes vitamin A, vitamin D, vitamin E, or a combination thereof.

In some embodiments, the oil includes fatty acid. In some embodiments, the fatty acid includes saturated fatty acid, unsaturated fatty acid, or saturated and unsaturated fatty acid.

In one embodiment, the saturated fatty acid includes, but is not limited to, medium-chain saturated fatty acid (e.g., MASESTER™ E6000, medium chain triglycerides oil (MCT oil), caprylic triglyceride, capric triglyceride, Miglyol 812®, MCT category in the Captex®, or a combination thereof), long-chain saturated oil (e.g., coconut oil, palm oil, chicken fat, lard, tallow, or a combination thereof), saturated fatty acid ester (e.g., isopropyl myristate, propylene glycol dicaprylate/dicaprate (Miglyol 840®), glyceryl monocaprylate/monocaprate, or a combination thereof), or a combination thereof.

In one embodiment, the unsaturated fatty acid includes, but is not limited to, monounsaturated fatty acid (e.g., olive oil, peanut oil, sunflower oil, or a combination thereof), polyunsaturated fatty acid (e.g., corn oil, soybean oil, sesame oil, safflower oil, flaxseed oil, krill oil, or a combination thereof), gamma-linolenic acid (GLA) oil (e.g., evening primrose oil, borage oil, or a combination thereof), or a combination thereof.

In one embodiment, the saturated and unsaturated fatty acid includes, but is not limited to, grape seed oil, hemp seed oil, perilla oil, pumpkin seed oil, or a combination thereof.

The materials, methods, and examples in the present application are merely illustrative and are not intended to limit the present disclosure, unless otherwise stated. Although the present disclosure may be implemented or tested using methods or materials similar or equivalent to those described herein, those methods or materials described herein are preferred.

### Example 1: The Preparation Process of the Liquid Composition

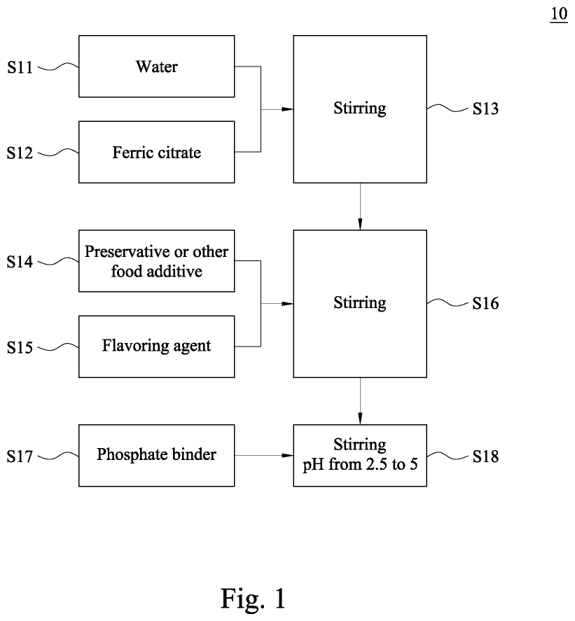

The flow chart in FIG. 1 illustrates the preparation process 10 of the liquid composition provided in the present application with the following steps: (1) at room temperature, water was provided in step S11 and ferric citrate was provided in step S12, and the water and the ferric citrate were mixed and stirred until dissolved in step S13 to obtain a first mixture; (2) a preservative or other food additives were provided in step S14, a flavoring agent was provided in step S15, and the preservative or other food additives and the flavoring agent were added into the first mixture and stirred until dissolved in step S16 to obtain a second mixture; (3) at least one phosphate binder was provided in step S17, and the at least one phosphate binder was added into the second mixture and stirred until dissolved in step S18, so that a pH of the liquid composition was stabilized at 2.5 to 5 to complete the preparation of the liquid composition. In subsequent examples, the effects of different components (e.g., flavoring agents or phosphate binders) on the phosphate binding capacity of ferric citrate were further tested.

### Example 2: The Selection of the Liquid Composition Formulation

Water-soluble flavoring agents typically contain propylene glycol, which is toxic to certain animals (such as cats or dogs). Therefore, oil-based flavoring agents were initially attempted to use. When the solvent of the liquid composition is water, an additional surfactant was necessary to be added when using an oil-based flavoring agent, so that the ferric citrate and the oil-based flavoring agent can be mixed thoroughly. Therefore, the present example first investigates the effect of the surfactant on the liquid composition and the phosphate binding capacity. The formulation is shown in Table 1. Wherein, the surfactant was first uniformly mixed with the flavoring agent to form an oil phase, and then added to the aqueous phase of the liquid composition. The effect of phosphate binding of each liquid composition was evaluated by adding potassium dihydrogen phosphate (KH₂PO₄) and visually observing whether a white phosphate precipitate of phosphate and ferric ions was formed (indicated by a positive reaction in the “phosphate binding experimental result” of Table 1). In this example, the concentrations of both ferric citrate and phosphate were 30 mM.

TABLE 1
Phosphate binding results of liquid compositions comprising
16% ferric citrate with different formulations

| | sample | | | | |
|---|---|---|---|---|---|
| | | F1471- | F1471- | F1471- | F1471- |
| formulation | control | 86-04 | 86-05 | 86-06 | 86-07 |
| ferric citrate | 16% | 16% | 16% | 16% | 16% |
| sodium citrate | — | 17.013% | 17.013% | 17.013% | 17.013% |
| oil-based | — | 0.05% | — | 0.05% | — |
| flavoring agent | | | | | |
| (beef flavoring | | | | | |
| agent) | | | | | |
| surfactant | — | 1.5% | — | — | 1.5% |
| (Tween 80) | | | | | |
| preservative | — | 0.066% | 0.066% | 0.066% | 0.066% |
| (potassium | | | | | |
| sorbate) | | | | | |
| phosphate | positive | positive | positive | positive | positive |
| binding | (1 min- | (1 hour) | (20 min- | (20 min- | (1 hour) |
| experimental | ute) | | utes) | utes) | |
| result | | | | | |

*Oil-based flavoring agent (beef flavoring agent): HENG YI TRADING CO., LTD.,

The effect of different formulations on the phosphate binding capacity was preliminarily determined by visually observing the time required for the binding of the ferric ions and the phosphate to form the white precipitate. The results showed that the addition of Tween 80 (surfactant) had a significant effect on phosphate binding (such as the comparison between the control and F1471-86-07), and the formation of a white precipitate was delayed, with the precipitate being observed only after 1 hour.

Therefore, to improve the effect of the phosphate binders and surfactants on phosphate binding capacity, the effects of using different phosphate binders and water-soluble flavoring agents (i.e., without surfactants) on phosphate binding capacity in liquid compositions comprising 10% ferric citrate were further tested. The formulations are shown in Table 2. Wherein, the concentration of ferric citrate in all of the liquid compositions was 5 mM, and the concentration of the phosphate was 30 mM. The experimental results were obtained by measuring the pH of the liquid compositions after standing, and visually observing the formation of phosphate precipitate. The groups marked “OK” represent that the phosphate precipitate was visually observed, indicating that the phosphate binding effect was achieved; while the groups marked “N/A” represent that the phosphate binding test was not performed.

TABLE 2
Phosphate binding results of liquid compositions comprising 10% ferric citrate with different formulations

| | sample No. (F1470-86-) | | | | | | | | | |
|---|---|---|---|---|---|---|---|---|---|---|
| formulation (%) | 1 | 2 | 3 | 4 | 7 | 8 | 9 | 10 | 11 | 12 |
| water | 86.63% | 78.88% | 86.68% | 79.88% | 79.88% | 86.58% | 70.88% | 87.88% | 86.13% | 79.88% |
| ferric citrate | 10.00% | 10.00% | 10.00% | 10.00% | 10.00% | 10.00% | 10.00% | 10.00% | 10.00% | 10.00% |
| Tween 80 | 1.00% | 1.00% | 1.00% | — | — | — | — | — | — | — |
| beef flavoring | 0.05% | 0.05% | 0.05% | — | — | — | — | — | — | — |
| agent | | | | | | | | | | |
| (oil-based) | | | | | | | | | | |
| meat flavoring | — | — | — | 0.05% | 0.05% | 0.05% | 0.05% | 0.05% | 0.05% | — |
| agent | | | | | | | | | | |
| (water-soluble) | | | | | | | | | | |
| meat flavoring | — | — | — | — | — | — | — | — | — | 0.05% |
| powder | | | | | | | | | | |
| (water-soluble) | | | | | | | | | | |
| potassium sorbate | 0.07% | 0.07% | 0.07% | 0.07% | 0.07% | 0.07% | 0.07% | 0.07% | 0.07% | 0.07% |
| sodium carbonate | 2.25% | — | — | — | — | — | — | — | — | — |
| sodium citrate | — | 10.01% | — | — | — | — | — | — | — | — |
| chitosan | — | — | 0.20% | — | — | — | — | — | — | — |
| lactic acid | — | — | 2.00% | — | — | — | — | — | — | — |
| calcium citrate | — | — | — | 10.01% | — | — | — | — | — | — |
| calcium glycinate | — | — | — | — | — | 3.30% | — | — | — | — |
| calcium lactate | — | — | — | — | 10.01% | — | — | — | — | 10.01% |
| magnesium sulfate | — | — | — | — | — | — | 19.01% | — | — | — |
| calcium carbonate | — | — | — | — | — | — | — | 2.00% | — | — |
| calcium acetate | — | — | — | — | — | — | — | — | 3.75% | — |
| total content | 100.00% | 100.00% | 100.00% | 100.00% | 100.00% | 100.00% | 100.00% | 100.00% | 100.00% | 100.00% |
| experimental | | | | | | | | | | |
| result | | | | | | | | | | |
| pH | 3.49 | 4.54 | (note 3) | (note 4) | 3.00 | 3.09 | 1.53 | 3.05 | 3.01 | 2.96 |
| phosphate binding | N/A | (note 2) | N/A | N/A | OK | OK | N/A | OK | OK | OK |
| effect | (note 1) | | | | | | | | | |

(note 1):
The solution foamed and was unstable; thus, no further experiments were conducted.
(note 2):
Binding time was delayed.
(note 3):
Insoluble; thus, no further experiments were conducted.
(note 4):
Insoluble; thus, no further experiments were conducted.
*Beef flavoring agent (oil-based): HENG YI TRADING CO., LTD., FAT163220.
*Meat flavoring agent (water-soluble): HENG YI TRADING CO., LTD., FAT152763.
*Meat flavoring powder (water-soluble): SHINEMATE CO., LTD., SM-1604.

The effect of each formulation on the binding of phosphate in the liquid composition was determined by visually observing the white phosphate precipitate. Regarding the effect of flavoring agents, the results showed that the use of water-soluble flavoring agent (F1470-86-12) of meat flavoring powder (SHINEMATE CO., LTD) did not affect the phosphate binding capacity of the liquid composition. Furthermore, the use of propylene glycol-free meat flavoring powder may avoid toxicity to animals such as cats or dogs, while achieving the desired flavoring effect.

Regarding the effect of the phosphate binders, a specific weight ratio of the ferric citrate and the phosphate binders was used in each group of samples, and the phosphate precipitates was observed in groups of F1470-86-07 (using calcium lactate), F1470-86-08 (using calcium glycinate), F1470-86-10 (using calcium carbonate), F1470-86-11 (using calcium acetate), and F1470-86-12 (using calcium lactate), which exhibited phosphate-binding capacity. Moreover, the pH of the prepared liquid compositions of these groups were stabilized at 2.5 to 5, wherein the phosphate binders used in these groups may be options for the phosphate binder. Phosphate binders also have the effect of adjusting the pH.

Regarding the phosphate binders, the present example further tested the phosphate binding results liquid compositions comprising 5% and 1% ferric citrate. Formulations are shown in Table 3 and Table 4. Similarly, different concentrations of the ferric citrate in combination with the phosphate binders, such as calcium lactate, calcium glycinate, calcium carbonate, and calcium acetate, showed great effects of phosphate binding. In summary, a pH of the liquid composition is stabilized at 2.5 to 5 and exhibits excellent phosphate-binding capacity by using specific phosphate binders and adjusting the ferric citrate/the phosphate binder to a specific weight ratio.

TABLE 3
Phosphate binding results of liquid compositions comprising
5% ferric citrate with different formulations

| formulation | sample No. (F1481-86-) | | | |
|---|---|---|---|---|
| (%) | 1 | 2 | 3 | 4 |
| water | 89.88% | 93.17% | 93.87% | 93.08% |
| ferric citrate | 5.00% | 5.00% | 5.00% | 5.00% |
| meat flavoring agent | 0.05% | 0.05% | 0.05% | 0.05% |
| (water-soluble) | | | | |
| potassium sorbate | 0.07% | 0.07% | 0.07% | 0.07% |
| calcium glycinate | — | 1.71% | — | — |
| calcium lactate | 5.01% | — | — | — |
| calcium carbonate | — | — | 1.02% | — |
| calcium acetate | — | — | — | 1.80% |
| total content | 100.00% | 100.00% | 100.00% | 100.00% |
| experimental result | | | | |
| pH | 3.14 | 3.19 | 3.90 | 3.16 |
| phosphate binding | OK | OK | OK | OK |
| effect | | | | |

* Groups marked “OK” represent that the phosphate precipitate was visually observed.

TABLE 4
Phosphate binding results of liquid compositions comprising
1% ferric citrate with different formulations

| formulation | sample No. (F1481-86-) | | | |
|---|---|---|---|---|
| (%) | 1 | 2 | 3 | 4 |
| water | 97.88% | 98.58% | 98.68% | 98.48% |
| ferric citrate | 1.00% | 1.00% | 1.00% | 1.00% |
| meat flavoring agent | 0.05% | 0.05% | 0.05% | 0.05% |
| (water-soluble) | | | | |
| potassium sorbate | 0.07% | 0.07% | 0.07% | 0.07% |
| calcium glycinate | — | 0.30% | — | — |
| calcium lactate | 1.01% | — | — | — |
| calcium carbonate | — | — | 0.20% | — |
| calcium acetate | — | — | — | 0.40% |
| total content | 100.00% | 100.00% | 100.00% | 100.00% |
| experimental result | | | | |
| pH | 3.46 | 3.27 | 4.61 | 3.95 |
| phosphate binding | OK | OK | OK | OK |
| effect | | | | |

* Groups marked “OK” represent that the phosphate precipitate was visually observed.

### Example 3: Test of the Phosphate Binding Capacity of the Liquid Compositions

Regarding the abovementioned examples, the liquid compositions (F1470-86-07, F1470-86-08, F1470-86-10, F1470-86-11, and F1470-86-12) that achieve a phosphate binding effect were further measured to determine their phosphate binding capacity. The steps were as follows: (1) 0.62 g of each liquid composition was acquired and respectively added to 50 mL of purified water; (2) 1.5 mL of 1 M potassium dihydrogen phosphate (KH₂PO₄) (Honeywell Fluka) was added to each liquid, respectively, so that the phosphate concentration in each liquid was 30 mM, and the pH of the solution was adjusted to approximately 2 using 4N hydrochloric acid, followed by shaking at 75 revolutions per minute for 2 hours in a 37° C. water bath to ensure the completion of phosphate reaction in the solution; (3) after the reaction, the solution was centrifuged at 6000 revolutions per minute for 15 minutes, and the supernatant was collected and diluted with water so that the phosphate concentration of each samples was within the range of the standard solution curve to obtain the samples to be tested; (4) Abnova phosphate assay kit (Colorimetric) Component A: 1 mM phosphate standard solution was used and diluted with purified water, and prepared to seven standard solutions with different phosphate concentrations: 0 μM, 0.78125 UM, 1.5625 UM, 3.125 UM, 6.25 UM, 12.5 μM, and 25 μM. 400 μL of each standard solution and each sample to be tested were acquired, 100 μL of Component B:

MG Plus Reagent was added, and reacted for 30 minutes; (5) a spectrophotometer was used to measure the absorbance of each standard solution and the samples to be tested at a wavelength of 650 nm. A concentration-absorbance calibration curve was constructed according to the absorbance of the standard solutions. After the phosphate concentration of each sample to be tested was obtained according to the calibration curve, the amount of the phosphate precipitates in the liquid composition of each group was calculated. The ratio of the amount of phosphate precipitates to the total amount of iron in each group was further determined.

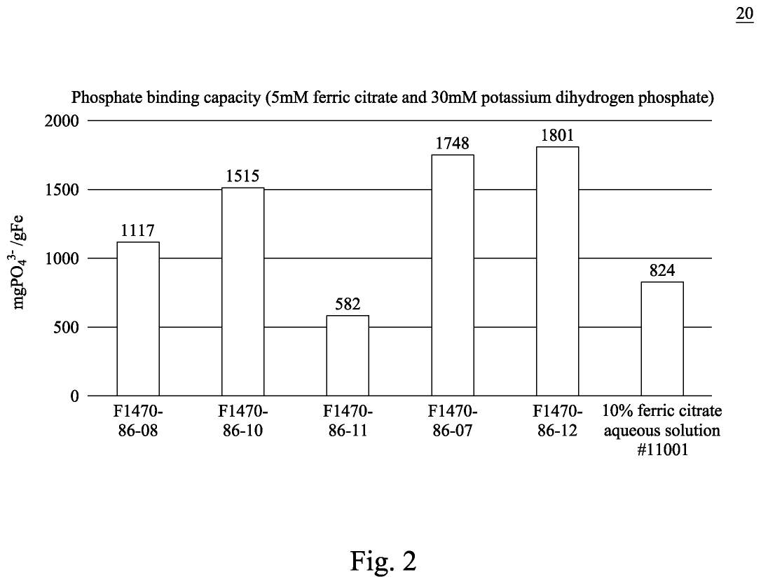

The phosphate binding results of liquid compositions F1470-86-07 (using calcium lactate), F1470-86-08 (using calcium glycinate), F1470-86-10 (using calcium carbonate), F1470-86-11 (using calcium acetate), F1470-86-12 (using calcium lactate), the control group (10% ferric citrate aqueous solution, containing 10% ferric citrate #11001 and water) are shown in drawing 20 in FIG. 2. The results show that when calcium lactate was used as the phosphate binder, the liquid composition exhibited the best phosphate binding effect, followed by calcium carbonate, calcium glycinate, and calcium acetate, sequentially.

### Example 4: Test of the Phosphate Binding Capacity of the Phosphate Binders

The results of Example 3 demonstrate that when calcium lactate was used as a phosphate binder, the liquid composition had the best phosphate binding capacity. Therefore, the present example further discussed the phosphate binding capacity of phosphate binders in liquid compositions. In this example, the binding capacity of each one of phosphate binders with phosphate was measured by simply mixing phosphate with each one of the phosphate binders without ferric citrate. The steps were as follows: (1) 0.62 g of liquid compositions containing calcium lactate, calcium glycinate, calcium carbonate, or calcium acetate (except for the absence of ferric citrate, the other formulations were the same as those of F1470-86-08, F1470-86-10, F1470-86-11, and F1470-86-12 in Example 3) and a control group (excluding ferric citrate and the phosphate binder, the other formulations were the same as those of F1470-86-12 in Example 3) were acquired, 50 mL of purified water was added, respectively, so that each sample had the same weight percentage of the phosphate binder as that of the samples described in the test of phosphate binding capacity in Example 3. Through the same steps in Example 3, the content of the precipitated phosphate was detected and the ratio of the amount of the phosphate precipitates to the amount of the phosphate binder in each group was further determined.

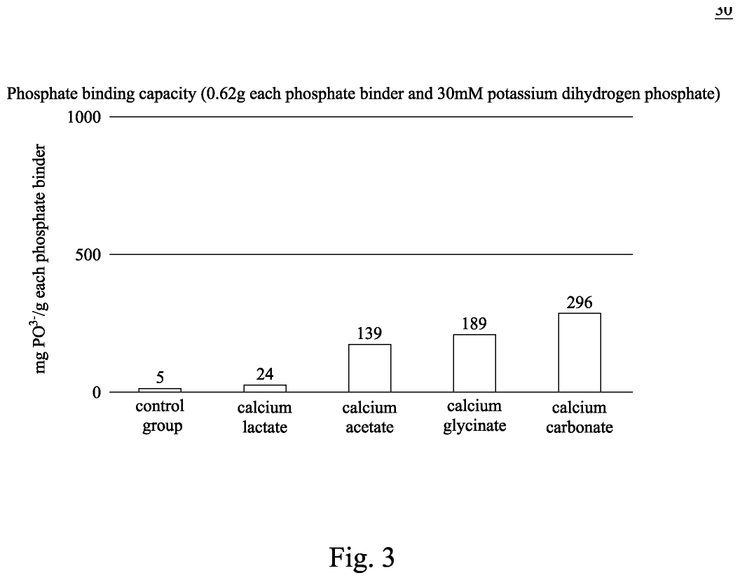

As shown in drawing 30 in FIG. 3, the experimental results indicate that calcium carbonate had the highest phosphate binding efficiency, followed by calcium glycinate, calcium acetate, and calcium lactate. Wherein, as Example 3 demonstrated, calcium lactate showed the best phosphate binding effect when being used as a phosphate binder. However, in this example, when calcium lactate was used alone, the binding effect with phosphate was the poorest among all phosphate binders. Therefore, the example shows that the phosphate binding capacity of the liquid composition was achieved through the interaction between ferric citrate and the phosphate binder. Furthermore, a synergistic effect is observed between ferric citrate and specific phosphate binders. For example, as shown in FIG. 2, the groups using calcium lactate, calcium carbonate, and calcium glycinate as phosphate binders all showed synergistic effects, giving the liquid composition of the present application an excellent phosphate binding capacity.

### Example 5: Test of the Phosphate Binding Capacity of the Phosphate Binders, the Ferric Citrate Solution, and the Liquid Compositions

In this example, the binding capacity of each group with phosphate was measured by mixing phosphate respectively with the phosphate binders (excluding ferric citrate), the ferric citrate solution, and the liquid composition. The steps were as follows: (1) phosphate binder group: 0.62 g of each liquid composition containing calcium lactate, calcium glycinate, calcium carbonate, or calcium acetate (the formulations were the same as F1470-86-12, F1470-86-08, F1470-86-10, F1470-86-11 in Example 3, except the absence of ferric citrate); ferric citrate solution group: 10% ferric citrate aqueous solution (#11001); liquid composition group: same as F1470-86-07, F1470-86-12, F1470-86-08, F1470-86-10, F1470-86-11 in Example 3 were acquired; (2) 50 mL of purified water was added to each group, so that each sample had the same weight percentage of the phosphate binders as that of the samples described in the test of phosphate binding capacity in Example 3, and by using the same steps in Example 3, the amount of the precipitated phosphate was detected and the ratio of the amount of the phosphate precipitates to the total amount of the weight of each group was further determined.

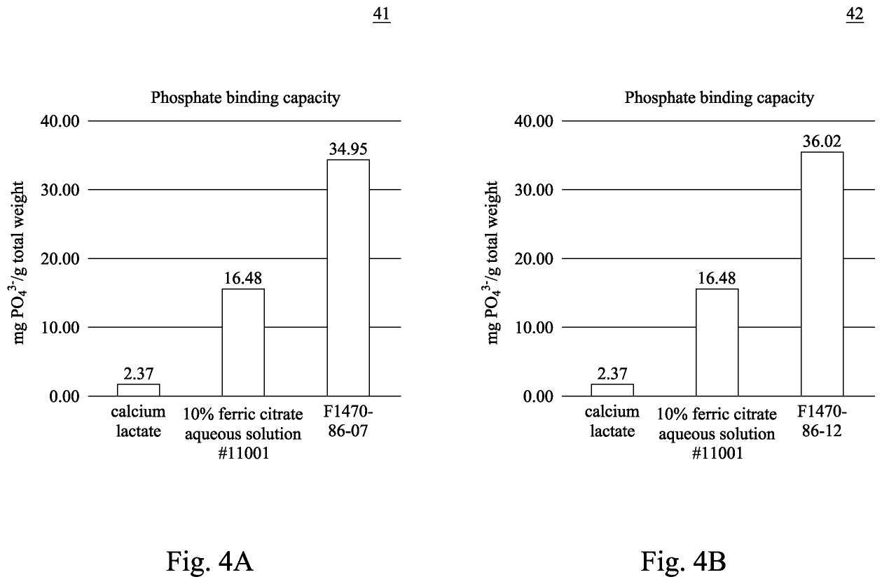

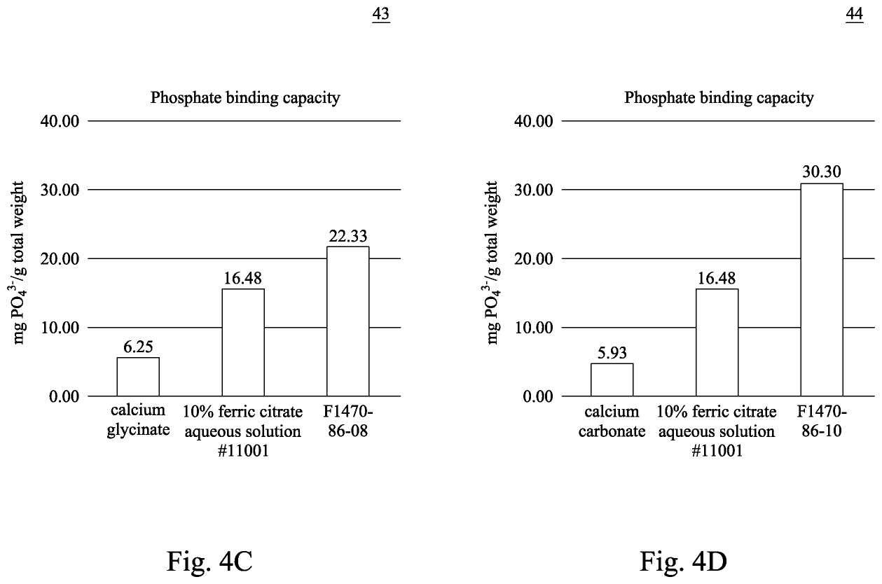

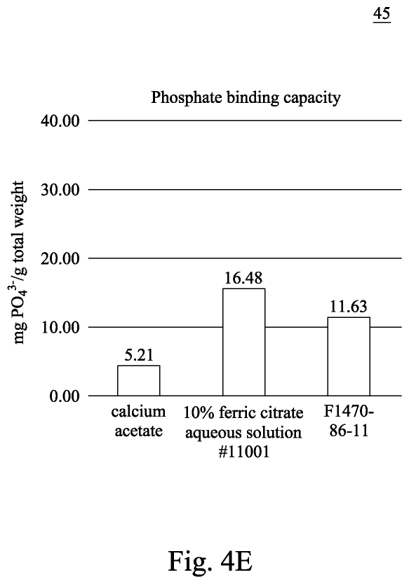

As shown in drawing 41 in FIG. 4A, drawing 42 in FIG. 4B, drawing 43 in FIG. 4C, drawing 44 in FIG. 4D, the experimental results indicate that when the phosphate binder in the liquid composition was selected form calcium lactate, calcium glycinate, and calcium carbonate, an unexpected synergistic effect was observed. In other words, the phosphate binding capacity of the liquid composition is achieved through the interaction between ferric citrate and the phosphate binder, and ferric citrate further produces a synergistic effect with specific phosphate binders.

Furthermore, as shown in drawing 45 in FIG. 4E, when calcium acetate was selected as a phosphate binder in the liquid composition, the phosphate binding capacity of the liquid composition was instead lower than that of 10% ferric citrate aqueous solution.

### Example 6: Test of Phosphate Binding Capacity of Oil Liquid Compositions

According to the aforementioned Example 4, a synergistic effect is produced between ferric citrate and calcium lactate, calcium glycinate, or calcium carbonate being used as phosphate binders, wherein the synergistic effect of calcium lactate as a phosphate binder was significant. In this example, the phosphate binding capacity of calcium lactate as a phosphate binder and ferric citrate in an oil was further tested. The phosphate concentration in this example was 30 mM.

In this example, an oil liquid composition F1519-2403 without vitamin E (including 84.95% of medium chain fatty acid (Masester™ E6000) (i.e., the oil in this example), 7.50% ferric citrate, 7.50% calcium lactate, and 0.05% crab flavoring agent (oil-based, from HENG YI TRADING CO., LTD., FAT248887)) and an oil liquid composition F1517-2404 with vitamin E (including 83.95% of medium chain fatty acid (Masester™ E6000), 7.50% ferric citrate, 7.50% calcium lactate, 0.05% crab flavoring agent (same as the crab flavoring agent in F1519-2403), and vitamin E (d1-alpha-tocopherol)) were stored under different temperature conditions (40° C. and 30° C.) for different periods of times (initial, stored for 1 month (1M), 2 months (2M), 3 months (3M), and 6 months (6M)), and the phosphate binding capacity of the oil liquid compositions (per mL) were tested for stability evaluation. The results were shown in Table 5 and Table 6, respectively. Regardless of whether vitamin E was contained, the oil liquid composition showed stable phosphate binding capacity after being stored at 30° C. and 40° C. for different periods of time.

TABLE 5
Phosphate binding results of the oil liquid composition
without vitamin E (F1519-2403) under different
storage time periods and conditions

storage condition

| storage time | 40° C./75% RH | 30° C./75% RH |
|---|---|---|
| initial | 27.26 mg | 27.26 mg |
| 1 M | 20.72 mg | 23.36 mg |
| 2 M | 30.01 mg | N/A |
| 3 M | 21.82 mg | 29.87 mg |
| 6 M | 22.3 mg | N/A |

*Groups marked “N/A” represent that the phosphate binding test was not performed.

TABLE 6
Phosphate binding results of the oil liquid composition with vitamin
E (F1519-2404) under different storage times and conditions

storage condition

| storage time | 40° C./75% RH | 30° C./75% RH |
|---|---|---|
| initial | 27.06 mg | 27.06 mg |
| 1 M | 25.36 mg | 25.33 mg |
| 2 M | 32.52 mg | N/A |
| 3 M | 19.81 mg | 24.18 mg |
| 6 M | 31.10 mg | N/A |

*Groups marked “N/A” represent that the phosphate binding test was not performed.

In the present example, an animal clinical trial of the oil liquid composition was further conducted. Animals with chronic kidney disease and hyperphosphatemia (01-006) were orally administered the oil liquid composition within 30 minutes after meals or with meals (either fed directly or mixed with food), and the changes in serum phosphorus levels were measured. The initial daily dose (mL) was calculated as body weight (Kg)*0.8 mL/Kg. At the week 4 follow-up visit, if the serum phosphorus level of the subject animal was <5.0 mg/dL, the daily dose was maintained at body weight (Kg)*0.8 mL/Kg; and if the serum phosphorus level of the subject animal was ≥5.0 mg/dl, the daily dose was adjusted to body weight (Kg)*1.2 mL/Kg. Furthermore, the daily dose of the oil liquid composition was divided equally among each meal of the subject animal. As shown in Table 7, at week 8 after daily administration of the oil liquid composition, the serum phosphorus level in the subject animal decreased (a reduction of 0.40 mg/dl compared to baseline).

TABLE 7
Changes in serum phosphorus levels (mg/dL) in the subject animal at
different times after administration of the oil liquid composition

time

| | day 0 | | |
|---|---|---|---|
| subject animal | (baseline) | week 4 | week 8 |
| 01-006 | 3.8 | 3.9 (+0.1) | 3.4 (−0.4) |

What is claimed is:

1. A liquid composition, comprising:
ferric citrate;
a phosphate binder; and
water;
wherein a weight ratio of the ferric citrate to the phosphate binder is from 6:1 to 0.9:1.

2. The liquid composition of claim 1, wherein the phosphate binder comprises a calcium salt of a weak acid.

3. The liquid composition of claim 2, wherein the calcium salt of the weak acid comprises: calcium lactate, calcium glycinate, calcium carbonate, calcium acetate, or a combination thereof.

4. The liquid composition of claim 1, wherein the ferric citrate and the phosphate binder have a synergistic effect.

5. The liquid composition of claim 3, wherein the ferric citrate and the calcium lactate have a synergistic effect.

6. The liquid composition of claim 1, further comprising a food additive, wherein the food additive comprises: a preservative, an antioxidant, a solubilizer, a flavoring agent, a thickener, or a combination thereof.

7. The liquid composition of claim 6, wherein the flavoring agent comprises a water-soluble flavoring agent.

8. The liquid composition of claim 6, wherein the flavoring agent is free of propylene glycol.

9. The liquid composition of claim 1, comprising 1% to 20% by weight of the ferric citrate, based on 100% by weight of the liquid composition.

10. The liquid composition of claim 1, comprising 1% to 15% by weight of the phosphate binder, based on 100% by weight of the liquid composition.

11. The liquid composition of claim 1, wherein a pH of the liquid composition is stabilized at 2.5 to 5.

12. A method for preventing or treating a disease in a subject, comprising administering to the subject an effective amount of the liquid composition of claim 1, wherein the disease comprises hyperphosphatemia, metabolic acidosis, or anemia.

13. The method of claim 12, wherein the subject comprises a human or an animal.

14. The method of claim 13, wherein the animal comprises a dog or a cat.

15. A liquid composition, comprising:
ferric citrate;
a phosphate binder; and
an oil;
wherein a weight ratio of the ferric citrate to the phosphate binder is from 6:1 to 0.9:1.

16. The liquid composition of claim 15, wherein the phosphate binder comprises a calcium salt of a weak acid.

17. The liquid composition of claim 16, wherein the calcium salt of the weak acid comprises: calcium lactate, calcium glycinate, calcium carbonate, calcium acetate, or a combination thereof.

18. The liquid composition of claim 16, further comprising vitamin A, vitamin D, vitamin E, or a combination thereof.

19. The liquid composition of claim 16, wherein the oil comprises fatty acid.
